(19) United States
(12) Patent Application Publication
(10) Pub. No.: US 2026/0097221 A1
(43) Pub. Date: Apr. 9, 2026

(54) SEQUENTIAL SHOCKS AS AN ANTIARRHYTHMIC THERAPY

(71) Applicant: Stryker Corporation, Portage, MI (US)

(72) Inventors: Tyson G. Taylor, Bothell, WA (US); Rose Tingwei Yin, Bellevue, WA (US); Fred W. Chapman, Newcastle, WA (US); Robert G. Walker, Seattle, WA (US); Daniel W. Piraino, Seattle, WA (US); David J. Linville, Redmond, WA (US); Ryan William Apperson, Bothell, WA (US)

(73) Assignee: Stryker Corporation, Portage, MI (US)

(21) Appl. No.: 19/352,368

(22) Filed: Oct. 7, 2025

Related U.S. Application Data
(60) Provisional application No. 63/705,482, filed on Oct. 9, 2024.

(51) Int. Cl. A61N 1/39; A61B 5/00; A61B 5/026; A61B 5/053; A61B 5/361; A61B 5/363; A61B 5/364; A61B 5/366

(52) U.S. Cl. CPC A61N 1/39044; A61B 5/026; A61B 5/053; A61B 5/361; A61B 5/363; A61B 5/364; A61B 5/366; A61B 5/4836; A61B 5/742; A61B 5/7475; A61N 1/3925; A61N 1/3987; A61N 1/3993

(57) ABSTRACT

An example method includes determining, before a subject has developed an arrhythmia or recurrence of the arrhythmia, that the subject is predicted to develop the arrhythmia. In response to determining that the subject is predicted to develop the arrhythmia, the example method further includes administering a sequence of external shocks to a heart of the subject, thereby preventing the subject from developing the arrhythmia or the recurrence of the arrhythmia.

### CROSS-REFERENCE TO RELATED APPLICATION

This application claims the priority of U.S. Provisional App. No. 63/705,482, which was filed on Oct. 9, 2024 and is incorporated by reference herein in its entirety.

### BACKGROUND

Various types of electrotherapies can be applied to treat cardiac arrhythmias. For example, the administration of pacing pulses is a treatment for bradycardia. In another example, the administration of an electrical shock is capable of treating certain arrhythmias, such as ventricular fibrillation (VF) and pulseless ventricular tachycardia (VT).

### BRIEF DESCRIPTION OF THE DRAWINGS

FIG. 1 illustrates an environment for administrating sequential shocks as an antiarrhythmic therapy.

FIGS. 2A to 2C illustrate examples of different types of sequential-shock treatments, which may be suitable for preventing the occurrence or recurrence of an arrhythmia when administered to a subject.

FIG. 3 illustrates an example process for preventing the occurrence or recurrence of an arrhythmia.

FIG. 4 illustrates an example process for determining whether an antiarrhythmic, sequential-shock therapy is appropriate.

FIG. 5 illustrates an example of an external defibrillator configured to perform various functions described herein.

FIGS. 6A and 6B illustrate examples of environments and timing related to administering a sequential-shock therapy. FIG. 6A shows an environment configured to administer the sequential-shock therapy. FIG. 6B shows a timing relationship of multiple shocks administered in the sequential-shock therapy.

### DETAILED DESCRIPTION

Various implementations described herein relate to sequential-shock therapies that are capable of preventing the occurrence, or recurrence, of cardiac arrhythmias. In some techniques described herein, an entity determines that a subject has a condition that is indicative of the future development of a cardiac arrhythmia. In response to that determination, the entity may recommend or administer a sequential-shock therapy that is capable of preventing the arrhythmia from developing. In various cases, the sequential-shock therapy includes the administration of more than two external electrical shocks (also referred to as “external shocks”) by one or more medical devices (e.g., defibrillators). The external electrical shocks may have different energy levels, durations, and vectors.

Unlike previous types of electrotherapies, various sequential-shock therapies described herein are administered before the instance of an arrhythmia occurs. In various cases, an example sequential-shock therapy includes external electrical shocks with a different timing relationship than conventional pacing pulses. In various examples, the example sequential-shock therapy includes external electrical shocks with a lower energy level than electrical shocks capable of defibrillation.

Implementations of the present disclosure will now be described with reference to the accompanying figures.

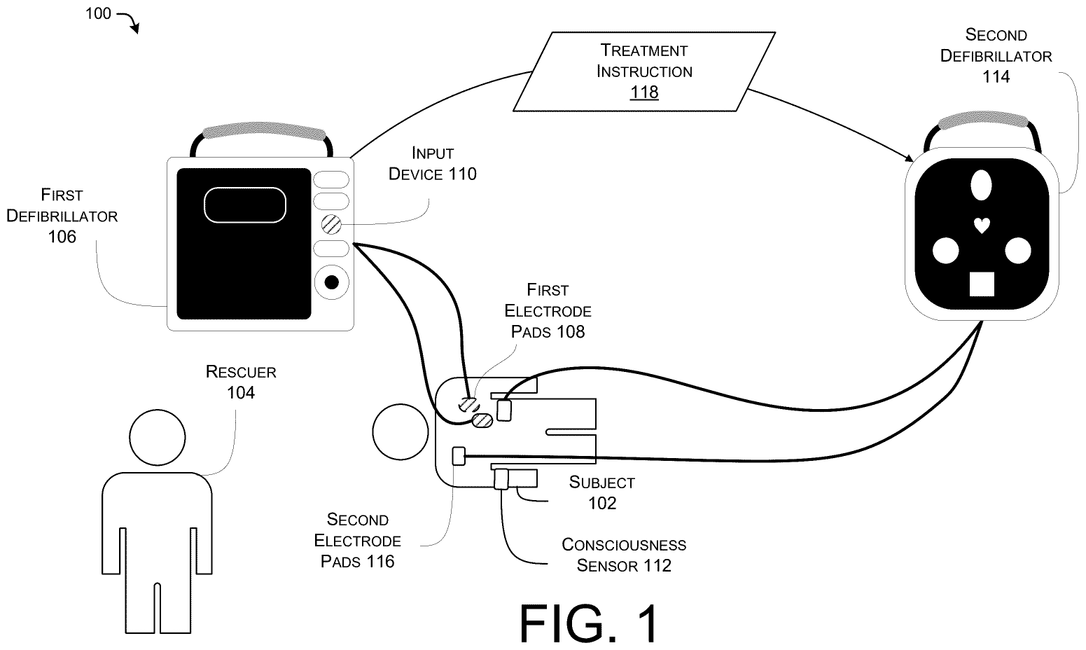

FIG. 1 illustrates an environment 100 for administrating sequential shocks as an antiarrhythmic therapy. In some cases, the environment 100 is a clinical environment, such as within a hospital, medical clinic, hospice, or other environment designed to provide medical care. In some examples, the environment 100 is a non-clinical environment. For example, the environment 100 may be a public space, such as an airport terminal, school, office building, home, or other environment in which individuals can experience sudden medical emergencies.

A subject 102 has a suspected medical condition. In some cases, the subject 102 presents with one or more symptoms of the medical condition, such as dizziness, nausea, loss-of-consciousness, seizure, or some other indication that the subject 102 has the medical condition. In various cases, the medical condition is, or is associated with, an arrhythmia. As used herein, the term “arrhythmia,” and its equivalents, can refer to an irregular heart rhythm.

In various cases, a rescuer 104 is deployed to the side of the subject 102 in response to the medical condition. In some examples, the rescuer 104 is a nurse, a physician, a physician's assistant, or some other person with specific clinical knowledge. In some cases, the rescuer 104 is an emergency medical services (EMS) professional. In some examples, the rescuer 104 has been deployed to the environment 100 in response to a report that the subject 102 is experiencing the medical condition. For instance, the rescuer 104 has been deployed to the environment 100 in response to a call indicating that the subject 102 has suddenly collapsed in the environment 100.

To monitor the medical condition of the subject 102, the rescuer 104 utilizes a first defibrillator 106. The first defibrillator 106, for instance, is a monitor-defibrillator. According to some cases, the first defibrillator 106 is a wearable defibrillator. In some cases, the first defibrillator 106 is a portable medical device. For example, the rescuer 104 carried the first defibrillator 106 to the environment 100 from a vehicle (e.g., an ambulance or other emergency response vehicle). The first defibrillator 106 is configured to detect one or more physiological parameters of the subject 102 via one or more accessory devices. Examples of physiological parameters include electrocardiogram (ECG), electroencephalogram (EEG), blood flow (e.g., instantaneous blood velocity, volumetric blood flow, etc.), pulse rate, heart rate, blood oxygenation (e.g., regional oxygenation, pulse oximetry, oxygen saturation, etc.), airway parameters (e.g., flow rate of air in the airway, a partial pressure of oxygen and/or carbon dioxide in the airway, airway pressure, etc.), blood pressure (e.g., pulse pressure, systolic blood pressure, diastolic blood pressure, mean arterial blood pressure, etc.), physiological sounds (e.g., heart sounds, breath sounds, etc.), and the like. The accessory device(s) include one or more sensors, such as one or more electrodes, an ultrasound transducer (e.g., configured to detect physiological structures and/or fluid movement using the Doppler effect), an oximetry sensor, a flow sensor, an oxygen sensor, a carbon dioxide sensor, a blood pressure sensor (e.g., a blood pressure cuff, catheter sensor, etc.), a microphone, an accelerometer, a gyroscope, and the like.

In particular cases, the first defibrillator 106 is electrically coupled with first electrode pads 108 disposed on the skin of the subject 102. For instance, the first electrode pads 108 are adhered to the skin on the chest and/or back of the subject 102. The first electrode pads 108, for example, extend along a first vector that intersects the heart of the subject 102. The first vector, for example, is an electrical path that passes through the body of the subject 102.

The first defibrillator 106 is configured to detect an ECG of the subject 102 using the first electrode pads 108. The ECG, for instance, is representative of an electrical signal output by the heart of the subject 102 over time. The ECG is detected as a voltage between electrodes within the first electrode pads 108, for instance.

In various cases, the medical condition of the subject 102 is in danger of worsening. For example, the medical condition of the subject 102 is indicative of a future instance of a dangerous arrhythmia. Without prophylactic treatment, the subject 102 may experience the arrhythmia in the future.

The rescuer 104 and/or the first defibrillator 106, in various cases, recognize one or more signs of a future occurrence or recurrence of the arrhythmia by monitoring one or more physiological parameters of the subject 102. In particular cases, the first defibrillator 106 predicts the occurrence or recurrence of the arrhythmia based on at least one of an ECG (e.g., a single lead, 3-lead, 12-lead, or any combination thereof), EEG, blood flow (e.g., instantaneous blood velocity, volumetric blood flow, etc.), pulse rate, heart rate, blood oxygenation (e.g., regional oxygenation, pulse oximetry, oxygen saturation, etc.), airway parameters (e.g., flow rate of air in the airway, a partial pressure of oxygen and/or carbon dioxide in the airway, airway pressure, etc.), blood pressure (e.g., pulse pressure, systolic blood pressure, diastolic blood pressure, mean arterial blood pressure, etc.), physiological sounds (e.g., heart sounds, breath sounds, etc.), or any combination thereof, of the subject 102. In some examples, the first defibrillator 106 predicts the future occurrence of a first arrhythmia (e.g., VF or VT) based on a present occurrence of a second arrhythmia. The second arrhythmia, for example, is a tachyarrhythmia. In various cases, the second arrhythmia includes a premature ventricular contraction (PVC), atrial fibrillation, supraventricular tachycardia, or the like. In various examples, the first defibrillator 106 predicts the occurrence or recurrence of the arrhythmia by determining that the parameter(s) of the subject are indicative of one or more conditions, such as previously observed course VF, other features associated with the ECG, ST elevation myocardial infarction (STEMI), occlusion myocardial infarction (OMI), atrial fibrillation, supraventricular tachycardia, excessive PVCs (e.g., more than a threshold number of PVCs in a time interval and/or more than a threshold number of consecutive PVCs), insufficient PVCs (e.g., less than a threshold number of PVCs in a time interval and/or less than a threshold number of consecutive PVCs), an AV block, or any combination thereof. Various techniques for classifying ECG are described, for instance, in the Physicians Guide to the Glasgow 12-lead ECG Analysis Program, published in 2009 by Physio-Control, Inc. of Redmond, WA, which is incorporated by reference herein in its entirety.

Coarse VF, in various cases, can be identified by determining that a peak amplitude of an ECG indicative of VF is above a threshold. In contrast, fine VF can be identified by determining that a peak amplitude of an ECG indicative of VF is below the threshold.

In some examples, the first defibrillator 106 predicts that a present VF rhythm is indicative of a future VF rhythm based on a rate of the present VF. For example, a high rate of VF is associated with shortened action potential duration, which can be proarrhythmic. In various cases, the first defibrillator 106 utilizes an AMSA, an amplitude, or peak frequency of a current VF rhythm in order to predict the occurrence of VF in the future. For example, the first defibrillator 106 may predict the future recurrence of VF by determining that a frequency of peaks in a current VF rhythm is above a first threshold frequency. In some cases, an excessively slow current VF rhythm may also be predictive of future VF recurrence. For example, the first defibrillator 106 may predict the future recurrence of VF by determining that the frequency of the peaks in the current VF rhythm is below a second threshold frequency, wherein the second threshold frequency is lower than the first threshold frequency.

In some cases, the first defibrillator 106 predicts the occurrence or recurrence of an arrhythmia based on the ECG of the subject 102 before and after the administration of a therapy. For example, the first defibrillator 106 compares a frequency, rate, AMSA, peak amplitude, or combination thereof, of the ECG before and after chest compressions are initiated on the subject 102. Changes in these parameters, for instance, may indicate that reperfusion due to the chest compressions has decreased action potential duration, such that the likelihood of a future occurrence or recurrence of VF is significantly higher. For example, if there is greater than a threshold change in the frequency, rate AMSA, peak amplitude, or metric representing a combination thereof, before and after the administration of chest compressions, then the first defibrillator 106 may predict the occurrence or recurrence of the arrhythmia.

In some examples, the first defibrillator 106 predicts the occurrence or recurrence of an arrhythmia based on a QT interval indicated by the ECG. In various cases, if at least one QT interval in the ECG is greater than a threshold, the first defibrillator 106 predicts the occurrence of the future arrhythmia (e.g., VF).

In various cases, STEMI and/or OMI can be predicted based, at least in part, on the ECG. According to some cases, the first defibrillator 106 predicts that the subject has a STEMI by determining that an ST segment of at least one lead the ECG is elevated above a baseline (e.g., a level of a TP segment). OMI, for instance, can be predicted based on determining that an ST segment of at least one lead in the ECG is elevated, determining that an ST segment of at least one lead in the ECG is depressed, identifying acute T-waves, identifying terminal QRS distortion, identifying that an amplitude of one or more QRS complexes is below a threshold, determining that a ratio of an ST interval with respect to a T wave amplitude is greater than a threshold, determining that a duration of a QRS complex is greater than a threshold, or a combination thereof.

The first defibrillator 106, for instance, detects one or more PVCs of the subject 102 by analyzing the ECG. A PVC is a type of heartbeat that is initiated by ventricular tissue (e.g., Purkinje fibers, his-Purkinje systems, ventricular myocytes, etc.), as opposed to a sinus or AV node of the heart. Unlike a QRS complex, a PVC generally omits a p-wave, a t-wave of the PVC is larger than a t-wave of a QRS complex, a polarity of the t-wave is opposite to the polarity of a t-wave of a QRS complex, a longer QT interval than that of QRS complexes, or a combination thereof. One or more of these characteristics can be identified in the ECG of the subject 102, for instance. In various cases, three or more consecutive PVCs, occurring at a rate that is greater than a threshold (e.g., 100 beats-per-minute), are classified as VT.

The first defibrillator 106, in various cases, detects atrial fibrillation of the subject 102 by analyzing the ECG. Atrial fibrillation is the result of disorganized electrical activity in the atria as well as atrial contraction. For instance, in some cases, an instance of atrial fibrillation lacks a p-wave and/or the presence of one or more QRS complexes occurring in an irregular heart rhythm (e.g., a timing between consecutive QRS complexes is inconsistent and/or non-repeating).

In various cases, the defibrillator 106 detects supraventricular tachycardia of the subject 102 by analyzing the ECG. Supraventricular tachycardia, in some cases, is indicated by narrow complexes in the ECG that occur at a relatively high rate. For example, supraventricular tachycardia is identified by determining that the ECG is indicative of greater than a threshold (e.g., 100) number of heartbeats in a threshold time period (e.g., a minute).

In various implementations, the defibrillator 106 identifies the AV block by analyzing the ECG. AV block refers to the interruption or delay of the transmission of electrical signals from the atria to the ventricles of the heart. In the ECG, the AV block is evidenced by a long PR interval (e.g., a PR interval longer than a threshold time period, such as 200 milliseconds (ms). The presence of AV block, in some cases, is predictive of slower rhythms that can result in PVCs and/or increase arrhythmogenicity.

In some implementations, the defibrillator 106 predicts the occurrence or recurrence of a particular arrhythmia by inputting, into a classifier, data indicative of the ECG. In some implementations, the classifier is a trained machine learning (ML) classifier. For example, the classifier is trained to identify predictive characteristics, in the ECG, that are indicative of the future occurrence or recurrence of the arrhythmia. In some cases, the classifier can be trained using a supervised learning technique, in which various parameters of the classifier are optimized to fit sample ECG segments of individuals who are later determined to develop the arrhythmia. Examples of suitable classifiers include, for instance, decision trees, support vector machines (SVMs), random forests, k-nearest neighbor (KNN) models, or any combination thereof.

One or more of the conditions described above can be identified based, at least in part, on non-ECG parameters. For example, various arrhythmias and other heart-relevant conditions are evidenced by light-headedness (e.g., identifying a blood flow, blood oxygenation, or blood pressure below a threshold), shortness-of-breath (e.g., identifying a respiratory rate above a threshold), abnormal heart sounds, or any combination thereof.

In some cases, the occurrence or recurrence of the arrhythmia is further identified based on one or more additional types of information. For example, the first defibrillator 106 may infer that the subject 102 has a condition associated with an increase susceptibility to developing the arrhythmia. According to some cases, the first defibrillator 106 predicts the occurrence or recurrence of the arrhythmia based, at least in part, on one or more demographics of the subject 102. For example, age, sex, and the use of an implantable electrotherapy device (e.g., an implantable pacemaker or implantable defibrillator). For example, the first defibrillator 106 determines that the occurrence or recurrence of the arrhythmia is greater if the subject 102 is young (e.g., under the age of 60), male, has an implantable electrotherapy device, or a combination thereof. In some cases, the first defibrillator 106 identifies the presence of the implantable electrotherapy device by receiving a communication signal from the device and/or detecting an artifact indicative of an electrical signal (e.g., electrical shock) output by the implantable electrotherapy device in the ECG of the subject 102. In some examples, the first defibrillator 106 predicts the occurrence or recurrence of the arrhythmia based, at least in part, on a patient history of the subject 102. For instance, the first defibrillator 106 receives an indication that the subject 102 has previously experienced the arrhythmia, such as in an electronic medical record (EMR) associated with the subject 102. In some cases, the first defibrillator 106 predicts the occurrence or recurrence of the arrhythmia based, at least in part, on the subject 102 having been previously diagnosed with Wolf-Parkinson-White syndrome.

In various implementations of the present disclosure, the first defibrillator 106 is configured to output a recommendation to administer an antiarrhythmic treatment to the rescuer 104 prior to the occurrence or recurrence of the arrhythmia.

In various cases, the first defibrillator 106 administers at least a portion of the antiarrhythmic treatment to the subject 102 in response to an input device 110 of the first defibrillator 106 receiving an input signal from the rescuer 104. In some cases, the input device 110 includes a button and/or touch sensor that detects pressing and/or touching of the input device 110 by the rescuer 104. In various cases, the rescuer 104 provides the input signal in response to perceiving the recommendation to administer the antiarrhythmic treatment.

In various implementations, the antiarrhythmic treatment includes a sequence of external electrical shocks administered to the heart of the subject 102. The external electrical shocks, for example, are administered through the skin of the subject 102. For instance, the first defibrillator 106 is configured to output one or more of the electrical shocks to electrodes within the first electrode pads 108.

In various cases, the antiarrhythmic treatment includes ten or fewer electrical shocks. In some cases, the antiarrhythmic treatment includes a continuous sequence of electrical shocks that are administered until the first defibrillator 106 receives another input signal from the rescuer 104 indicating a request to cease administration of the electrical shocks.

According to some instances, at least two of the electrical shocks in the antiarrhythmic treatment are temporally overlapping. For example, the leading edge of one shock may occur before the lagging edge of another shock. In some cases, the electrical shocks are administered sequentially, such that the leading edge of one shock occurs after the lagging edge of a previous shock. According to various implementations, a time interval (e.g., delay) between a leading edge of first shock among the sequence and a leading edge of a second shock among the sequence is in a range of 10 ms to 10,000 ms.

In examples in which the ECG of the subject 102 includes QRS complexes associated with an organized heart rhythm, one or more of the electrical shocks are synchronized (e.g., temporally overlap) QRS complexes. For example, the first defibrillator 106 determines the timing of the electrical shocks based at least in part on the ECG of the subject 102. In some instances in which the ECG of the subject 102 is indicative of a disorganized rhythm, the electrical shocks may have a predetermined timing relationship that is independent of the ECG of the subject 102.

In some cases, the electrical shocks are coordinated with chest compressions administered to the subject 102. For example, the subject 102 is receiving chest compressions from a mechanical chest compression device (not illustrated) that outputs an indication of the timing of the chest compressions to the first defibrillator 106. Based on this indication, the first defibrillator 106 coordinates the timing of the electrical shocks. In some cases, one or more of the electrical shocks are specifically timed for a release phase of the chest compressions (e.g., an active decompression phase or a time in which the mechanical chest compression device is not administering a downward force on the chest of the subject 102). In some examples, one or more of the electrical shocks are timed for a compression phase of the chest compressions (e.g., a time in which the mechanical chest compression device is administering a downward force on the chest of the subject 102).

The individual electrical shocks in the antiarrhythmic treatment, in various cases, have lower energy levels than an electrical shock suitable for defibrillation. For example, the electrical shocks may have energy levels that are lower than 200 Joules (J). In some cases, the electrical shocks have energy levels in a range of 10 J to 360 J, a range of 0.1 J/kilogram (kg) of patient mass to 4 J/kg, or the like. If the subject 102 is a child, for example, the electrical shocks have energy levels in a range of 1 J to 90 J. The electrical shocks may be administered at two or more different energy levels. In some cases, the electrical shocks are administered at the same energy level. The electrical shocks are administered at one or more vectors, for instance.

According to some cases, the first defibrillator 106 outputs at least a portion of the electrical shocks via one or more capacitors. For example, the first defibrillator 106 charges the capacitor(s) using an on-board power source (e.g., a battery) and outputs one or more of the electrical shocks by discharging the capacitor(s). In some cases, the first defibrillator 106 outputs one of the electrical shocks by completely discharging a single capacitor. In some examples, the first defibrillator 106 outputs multiple electrical shocks by sequentially and partially discharging a single capacitor. The capacitor(s) of the first defibrillator 106, for example, may also be utilized to output defibrillation shocks, in some cases.

Optionally, the first defibrillator 106 outputs one or more of the external electrical shocks further in response to confirming that the subject 102 is unconscious and/or obtunded. In various cases, the first defibrillator 106 receives a signal indicating that the subject 102 is unconscious and/or obtunded from a consciousness sensor 112. In some implementations, the consciousness sensor 112 includes an accelerometer configured to detect motion of the subject 102 and/or a gyroscope configured to detect an orientation of the subject 102. The first defibrillator 106, for instance, infers that the subject 102 is conscious by detecting greater than a threshold amount of motion (e.g., acceleration) of the subject 102 and/or by determining that the orientation of at least a portion of the subject 102 is within a threshold range. In some cases, the first defibrillator 106 infers motion of the subject 102 by detecting an artifact in a physiological signal detected by another sensor. According to some cases, the first defibrillator 106 determines that the subject 102 is unconscious by determining that there is an absence of an irregular motion artifact (e.g., a non-periodic artifact, which could be the result of ventilation or chest compressions) in data indicative of a detected physiological parameter of the subject 102. In some cases, the first defibrillator 106 itself includes a motion sensor (e.g., an accelerometer and/or gyroscope) configured to detect motion of the first defibrillator 106. For instance, the first defibrillator 106 may distinguish between motion caused by transporting the first defibrillator 106 and the subject 102 (e.g., motion of an ambulance carrying the subject 102 and the first defibrillator 106), compared to motion of the subject 102 associated with consciousness. By waiting until the subject 102 has lost consciousness, the first defibrillator 106 may prevent the subject 102 from perceiving pain associated with the antiarrhythmic treatment.

In some examples, the antiarrhythmic treatment is administered jointly by the first defibrillator 106 and a second defibrillator 114. The second defibrillator 114, in various cases, is electrically coupled to second electrode pads 116 disposed on skin of the subject 102. For example, the second electrode pads 116 are adhered to skin on the chest and/or back of the subject 102. For instance, the second electrode pads 116 are arranged along a second vector that is different than the first vector. Although not specifically illustrated in FIG. 1, in some cases, the second defibrillator 114 is electrically connected with the first electrode pads 108.

The second defibrillator 114 is a portable medical device, in some cases. In various examples, the second defibrillator 114 is an automated external defibrillator (AED). In some cases, the second defibrillator 114 is a monitor-defibrillator, a wearable defibrillator, or an implantable defibrillator. Although not illustrated in FIG. 1, in some examples, the second defibrillator 114 is integrated into a mechanical chest compression device that is configured to administer chest compressions to the subject 102. In some cases, the second defibrillator 114 is a defibrillator accessory without a standalone monitoring capability.

In the example of FIG. 1, the first defibrillator 106 coordinates administration of the electrical shocks with the second defibrillator 114 by transmitting a treatment instruction 118 to the second defibrillator 114. The treatment instruction 118, for instance, includes one or more communication signals instructing the second defibrillator 114 to administer one or more of the electrical shocks in the antiarrhythmic treatment. In some cases, the treatment instruction 118 indicates one or more times at which the electrical shock(s) are to be administered by the second defibrillator 114. According to some cases, the treatment instruction 118 causes the second defibrillator 114 to activate one or more protection circuits that prevent electrical shocks output by the first defibrillator 106 from being absorbed by the circuitry within the second defibrillator 114.

The second defibrillator 114 is configured to output one or more of the electrical shocks to the second electrode pads 116 in response to the treatment instruction 118. In some examples, the second defibrillator 114 is configured to output one or more of the electrical shocks to the first electrode pads 108 in response to the treatment instruction 118. The second defibrillator 114 includes one or more capacitors. For instance, the second defibrillator 114 is configured to output one or more of the electrical shocks by discharging the capacitor(s). In some cases, the second defibrillator 114 delivers one of the electrical shocks by fully discharging one of the capacitor(s). In some examples, the second defibrillator 114 outputs multiple instances of the electrical shocks by partially discharging the one of the capacitor(s). In some implementations, the first defibrillator 106 and the second defibrillator 114 output alternating electrical shocks in the sequence of external electrical shocks.

In various cases, the antiarrhythmic therapy administered to the subject 102 prevents the subject 102 from developing the predicted arrhythmia. Accordingly, implementations of the present disclosure prevent the subject 102 from developing or redeveloping a harmful medical condition. For example, in some implementations, an antiarrhythmic treatment is administered to the subject 102 that prevents the subject 102 from developing VF or some other dangerous arrhythmia (e.g., AF). In some cases, the antiarrhythmic treatment delays the occurrence or recurrence of VF or the other dangerous arrhythmia. Notably, any delays in the occurrence or recurrence of a dangerous arrhythmia can reduce the burden of the arrhythmia on the body of the subject 102. For example, the severity of some injuries to the subject 102 as a result of an arrhythmia are proportional to the amount of time that the subject 102 exhibits the arrhythmia. Therefore, even delays to the occurrence or recurrence of the arrhythmia can have significant benefits to the health of the subject 102.

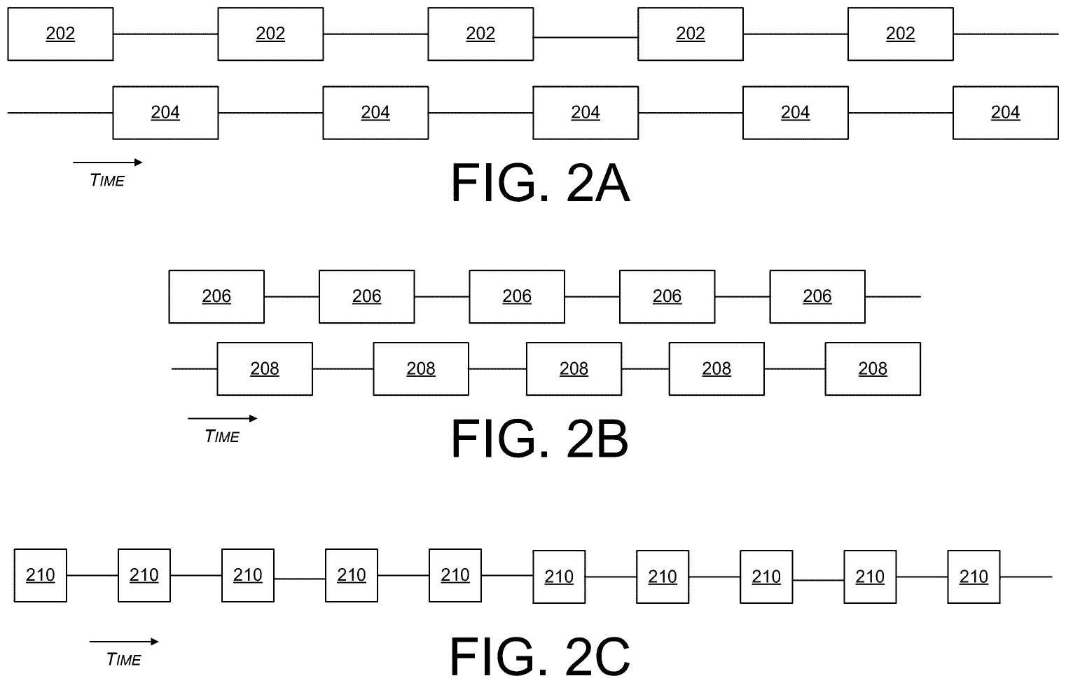

FIGS. 2A to 2C illustrate examples of different types of sequential-shock treatments, which may be suitable for preventing the occurrence or recurrence of an arrhythmia when administered to a subject.

FIG. 2A illustrates a first example of a sequential-shock treatment including nonoverlapping shocks administered by two different defibrillators. First shocks 202 in the sequential-shock treatment are output by a first defibrillator. Second shocks 204 in the sequential shock treatment are output by a second defibrillator. In various cases, the first defibrillator outputs the first shocks 202 by discharging at least one capacitor. In some examples, the first defibrillator at least partially recharges the capacitor(s) when the second defibrillator is outputting the second shocks 204.

FIG. 2B illustrates a second example of a sequential-shock treatment including overlapping shocks administered by two different defibrillators. First shocks 206 in the sequential-shock treatment are output by a first defibrillator. Second shocks 208 in the sequential shock treatment are output by a second defibrillator. For example, leading edges of the second shocks 208 occur before the lagging edges of the first shocks 206, such that the first shocks 206 and the second shocks 208 are temporally overlapping.

FIG. 2C illustrates a third example of a sequential shock treatment including shocks 210 administered by a single defibrillator. In various cases, the single defibrillator outputs the shocks 210 by discharging at least one capacitor. In some examples, the single defibrillator outputs the individual shocks 210 by partially discharging a single capacitor, such that multiple shocks can be delivered from a single charge of the single capacitor.

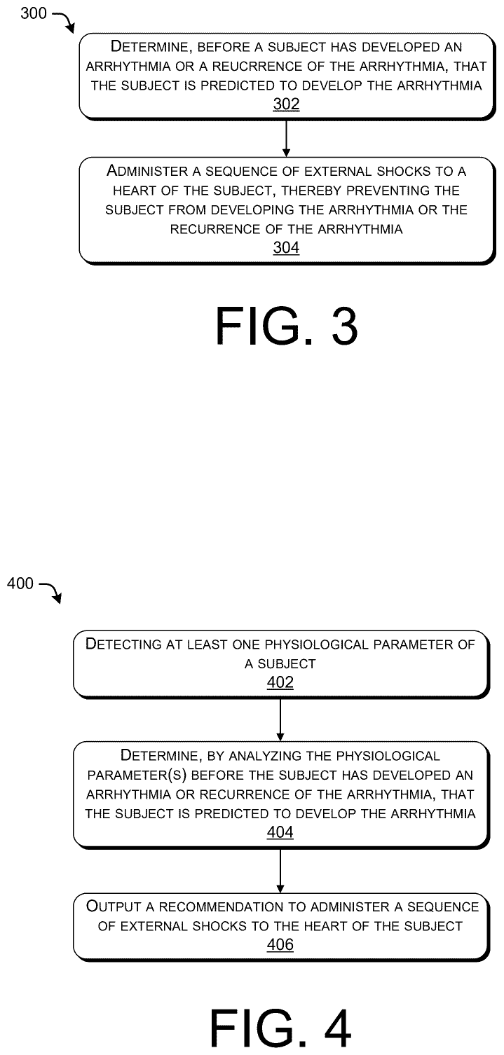

FIG. 3 illustrates an example process 300 for preventing the occurrence or recurrence of an arrhythmia. The process 300 is performed by an entity including a defibrillator (e.g., the first defibrillator 106 or the second defibrillator 114), multiple defibrillators (e.g., the first defibrillator 106 and the second defibrillator 114), a medical device, at least one processor, a computing device, or any combination thereof.

At 302, the entity determines, before a subject has developed an arrhythmia or a recurrence of the arrhythmia, that the subject is predicted to develop the arrhythmia. The arrhythmia, for instance, is at least one of VF, VT, a PVC, atrial fibrillation, or supraventricular tachycardia.

In some cases, the entity determines that the subject is predicted to develop the arrhythmia by determining that the subject has a condition that is predictive of the arrhythmia. In some cases, the condition includes another arrhythmia or other cardiac abnormality. For instance, the condition includes one or more of Wolf-Parkinson-White syndrome, coarse VF, STEMI, OMI, atrial fibrillation, supraventricular tachycardia, excessive PVCs, insufficient PVCs, or an AV block. In some examples, the entity determines that the subject has the condition by analyzing one or more physiological parameters of the subject, such as an ECG, blood flow, or transthoracic impedance. In some cases, the entity determines that the subject has the condition based on one or more demographics and/or a patient history of the subject. In some examples, the entity determines that the subject has previously had the condition, even if the condition is subsequently resolved.

At 304, the entity administers a sequence of external shocks to a heart of the subject, thereby preventing the subject from developing the arrhythmia or the recurrence of the arrhythmia. According to some cases, the sequence of external shocks includes 2 to 10 external shocks. The external shocks are administered at one or more energy levels, one or more vectors, or a combination thereof. For instance, the external shocks may each have an energy level in a range of 50 J to 360 J (e.g., if the subject is an adult). If the subject is a pediatric patient, for instance, the external shocks may each have an anergy level in a range of 1 J to 90 J.

The sequence of external shocks may have one or more timing relationships. In some cases, one or more of the external shocks are synchronized (e.g., temporally overlapping) with one or more QRS complexes in an ECG of the subject. If the ECG lacks QRS complexes, then the external shocks can be administered via a predetermined timing relationship. In some cases, a delay between a leading edge of a first external shock among the sequence and a leading edge of a second external sock among the sequence is in a range of 10 ms to 10,000 ms. One or more of the external shocks are temporally overlapping with at least one other external shock in the sequence, in some cases. According to some cases, one or more of the external shocks are administered during one or more release phases of chest compressions administered to the subject. In some examples, one or more of the external shocks are administered during one or more compression phases of the chest compressions.

One or more defibrillators are configured to administer the sequence of external shocks. The defibrillator(s) include one or more external defibrillators, a wearable defibrillator, an implantable defibrillator, or a combination thereof. In some cases, a first external shock is output by a first defibrillator, a second external shock is output by a second defibrillator, and a third external shock is output by the first defibrillator. Between administration of the first and third external shocks, a capacitor of the first defibrillator may be recharged, for instance. In some cases, the first defibrillator outputs the third external shock in response to recharging the capacitor. In some cases, a single defibrillator outputs multiple external shocks in the sequence by partially discharging a single capacitor. In some examples, a single defibrillator outputs multiple external shocks while recharging its capacitor between shocks.

Optionally, the sequence of external shocks is administered in response to one or more additional events. In some cases, the sequence of external shocks is administered in response to determining that the subject is unconscious. In some examples, the sequence of external shocks is administered in response to outputting a recommendation to administer the sequence and/or receiving an input signal from a user.

FIG. 4 illustrates an example process 400 for determining whether an antiarrhythmic, sequential-shock therapy is appropriate. The process 400 is performed by an entity including a defibrillator (e.g., the first defibrillator 106 or the second defibrillator 114), multiple defibrillators (e.g., the first defibrillator 106 and the second defibrillator 114), a medical device, at least one processor, a computing device, or any combination thereof.

At 402, the entity detects at least one physiological parameter of a subject. For example, the entity detects at least one of an ECG, blood flow, or transthoracic impedance. The entity, for instance, includes one or more sensors configured to detect the physiological parameter(s).

At 404, the entity determines, by analyzing the physiological parameter(s) before the subject has developed an arrhythmia or recurrence of the arrhythmia, that the subject is predicted to develop the arrhythmia. The arrhythmia, for instance, is at least one of VF, VT, a PVC, atrial fibrillation, or supraventricular tachycardia.

In some cases, the entity determines that the subject is predicted to develop the arrhythmia by determining that the subject has a condition that is predictive of the arrhythmia.

In some cases, the condition includes another arrhythmia or cardiac abnormality. For instance, the condition includes one or more of Wolf-Parkinson-White syndrome, coarse VF, STEMI, OMI, atrial fibrillation, supraventricular tachycardia, excessive PVCs, insufficient PVCs, or an AV block. In some examples, the entity determines that the subject has the condition by analyzing the physiological parameter(s). In some cases, the entity determines that the subject has the condition based on one or more demographics and/or a patient history of the subject. In some examples, the entity determines that the subject has previously had the condition, even if the condition is subsequently resolved.

At 406, the entity output a recommendation to administer a sequence of external shocks to the heart of the subject. For example, the entity outputs the recommendation via at least one output device. According to some cases, the sequence of external shocks includes 2 to 10 external shocks. The external shocks are administered at one or more energy levels, one or more vectors, or a combination thereof. For instance, the external shocks may each have an energy level in a range of 50 J to 360 J if the subject is an adult, or may each have an energy level in a range of 1 J to 90 J if the subject is a child.

The sequence of external shocks may have one or more timing relationships. In some cases, one or more of the external shocks are synchronized (e.g., temporally overlapping) with one or more QRS complexes in an ECG of the subject. If the ECG lacks QRS complexes, then the external shocks can be administered via a predetermined timing relationship. In some cases, a delay between a leading edge of a first external shock among the sequence and a leading edge of a second external sock among the sequence is in a range of 10 ms to 10,000 ms. One or more of the external shocks are temporally overlapping with at least one other external shock in the sequence, in some cases. According to some cases, one or more of the external shocks are administered during one or more release phases of chest compressions administered to the subject. In some examples, one or more of the external shocks are administered during one or more compression phases of the chest compressions.

One or more defibrillators are configured to administer the sequence of external shocks. The defibrillator(s) include one or more external defibrillators, a wearable defibrillator, an implantable defibrillator, or a combination thereof. In some cases, a first external shock is output by a first defibrillator, a second external shock is output by a second defibrillator, and a third external shock is output by the first defibrillator. Between administration of the first and third external shocks, a capacitor of the first defibrillator may be recharged, for instance. In some cases, the first defibrillator outputs the third external shock in response to recharging the capacitor. In some cases, a single defibrillator outputs multiple external shocks in the sequence by partially discharging a single capacitor. In some examples, a single defibrillator outputs multiple external shocks while recharging its capacitor between shocks.

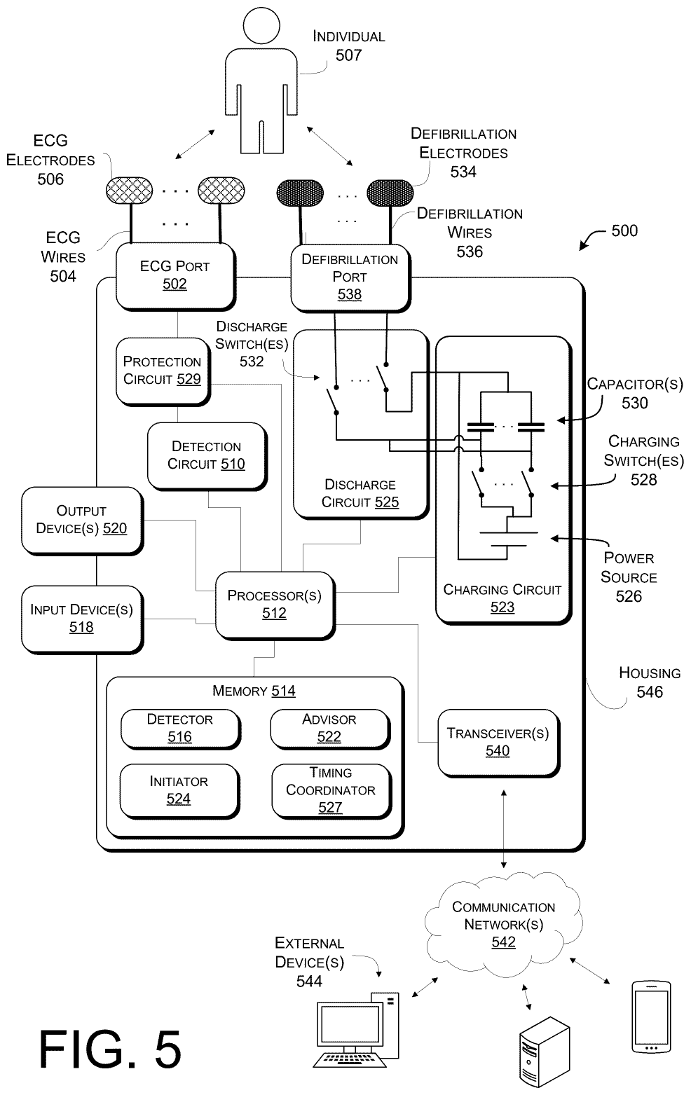

FIG. 5 illustrates an example of an external defibrillator 500 configured to perform various functions described herein. For example, the external defibrillator 500 is the first defibrillator 106 or secondary defibrillator 106 described above with reference to FIG. 1.

The external defibrillator 500 includes an electrocardiogram (ECG) port 502 connected to multiple ECG wires 504. In some cases, the ECG wires 504 are removeable from the ECG port 502. For instance, the ECG wires 504 are plugged into the ECG port 502 via connectors. The ECG wires 504 are connected to ECG electrodes 506, respectively. In various implementations, the ECG electrodes 506 are disposed on different locations on an individual 508. A detection circuit 510 (also referred to as a “measurement circuit”) is configured to detect relative voltages between the ECG electrodes 506. These voltages are indicative of the electrical activity of the heart of the individual 508.

In various implementations, the ECG electrodes 506 are in contact with the different locations on the skin of the individual 508. In some examples, a first one of the ECG electrodes 506 is placed on the skin between the heart and right arm of the individual 508, a second one of the ECG electrodes 506 is placed on the skin between the heart and left arm of the individual 508, and a third one of the ECG electrodes 506 is placed on the skin between the heart and a leg (either the left leg or the right leg) of the individual 508. In these examples, the detection circuit 510 is configured to measure the relative voltages between the first, second, and third ECG electrodes 506. Respective pairings of the ECG electrodes 506 are referred to as “leads,” and the voltages between the pairs of ECG electrodes 506 are known as “lead voltages. ” In some examples, more than three ECG electrodes 506 are included, such that 5-lead or 12-lead ECG signals are detected by the detection circuit 510.

The detection circuit 510 includes at least one analog circuit, at least one digital circuit, or a combination thereof. The detection circuit 510 receives the analog electrical signals from the ECG electrodes 506, via the ECG port 502 and the ECG wires 504. In some cases, the detection circuit 510 includes one or more analog filters configured to filter noise and/or artifact from the electrical signals. The detection circuit 510 includes an analog-to-digital (ADC) in various examples. The detection circuit 510 generates a digital signal indicative of the analog electrical signals from the ECG electrodes 506. This digital signal can be referred to as an “ECG signal” or an “ECG.”

In some cases, the detection circuit 510 further detects an electrical impedance between at least one pair of the ECG electrodes 506. For example, the detection circuit 510 includes, or otherwise controls, a power source that applies a known voltage (or current) across a pair of the ECG electrodes 506 and detects a resultant current (or voltage) between the pair of the ECG electrodes 506. The impedance is generated based on the applied signal (voltage or current) and the resultant signal (current or voltage). In various cases, the impedance corresponds to respiration of the individual 508, chest compressions performed on the individual 508, and other physiological states of the individual 508. In various examples, the detection circuit 510 includes one or more analog filters configured to filter noise and/or artifact from the resultant signal. The detection circuit 510 generates a digital signal indicative of the impedance using an ADC. This digital signal can be referred to as an “impedance signal” or an “impedance.”

The detection circuit 510 provides the ECG signal and/or the impedance signal one or more processors 512 in the external defibrillator 500. In some implementations, the processor(s) 512 includes a central processing unit (CPU), a graphics processing unit (GPU), both CPU and GPU, or other processing unit or component known in the art.

The processor(s) 512 is operably connected to memory 514. In various implementations, the memory 514 is volatile (such as random access memory (RAM)), non-volatile (such as read only memory (ROM), flash memory, etc.) or some combination of the two. The memory 514 stores instructions that, when executed by the processor(s) 512, causes the processor(s) 512 to perform various operations. In various examples, the memory 514 stores methods, threads, processes, applications, objects, modules, any other sort of executable instruction, or a combination thereof. In some cases, the memory 514 stores files, databases, or a combination thereof. In some examples, the memory 514 includes, but is not limited to, RAM, ROM, electrically erasable programmable read-only memory (EEPROM), flash memory, or any other memory technology. In some examples, the memory 514 includes one or more of CD-ROMs, digital versatile discs (DVDs), content-addressable memory (CAM), or other optical storage, magnetic cassettes, magnetic tape, magnetic disk storage or other magnetic storage devices, or any other medium which can be used to store the desired information and which can be accessed by the processor(s) 512 and/or the external defibrillator 500. In some cases, the memory 514 at least temporarily stores the ECG signal and/or the impedance signal.

In various examples, the memory 514 includes a detector 516, which causes the processor(s) 512 to determine, based on the ECG signal and/or the impedance signal, whether the individual 508 is exhibiting a particular heart rhythm. For instance, the processor(s) 512 determines whether the individual 508 is experiencing a shockable rhythm that is treatable by defibrillation. Examples of shockable rhythms include VF and ventricular tachycardia (V-Tach). In some examples, the processor(s) 512 determines whether any of a variety of different rhythms (e.g., asystole, sinus rhythm, atrial fibrillation (AF), etc.) are present in the ECG signal.

The processor(s) 512 is operably connected to one or more input devices 518 and one or more output devices 520. Collectively, the input device(s) 518 and the output device(s) 520 function as an interface between a user and the defibrillator 500. The input device(s) 518 is configured to receive an input from a user and includes at least one of a keypad, a cursor control, a touch-sensitive display, a voice input device (e.g., a microphone), a haptic feedback device (e.g., a gyroscope), or any combination thereof. The output device(s) 520 includes at least one of a display, a speaker, a haptic output device, a printer, or any combination thereof. In various examples, the processor(s) 512 causes a display among the input device(s) 518 to visually output a waveform of the ECG signal and/or the impedance signal. In some implementations, the input device(s) 518 includes one or more touch sensors, the output device(s) 520 includes a display screen, and the touch sensor(s) are integrated with the display screen. Thus, in some cases, the external defibrillator 500 includes a touchscreen configured to receive user input signal(s) and visually output physiological parameters, such as the ECG signal and/or the impedance signal.

In some examples, the memory 514 includes an advisor 522, which, when executed by the processor(s) 512, causes the processor(s) 512 to generate advice and/or control the output device(s) 520 to output the advice to a user (e.g., a rescuer). In some examples, the processor(s) 512 provides, or causes the output device(s) 520 to provide, an instruction to perform CPR on the individual 508. In some cases, the processor(s) 512 evaluates, based on the ECG signal, the impedance signal, or other physiological parameters, CPR being performed on the individual 508 and causes the output device(s) 520 to provide feedback about the CPR in the instruction. According to some examples, the processor(s) 512, upon identifying that a shockable rhythm is present in the ECG signal, causes the output device(s) 520 to output an instruction and/or recommendation to administer a defibrillation shock to the individual 508.

The memory 514 also includes an initiator 524 which, when executed by the processor(s) 512, causes the processor(s) 512 to control other elements of the external defibrillator 500 in order to administer a defibrillation shock to the individual 508. In some examples, the processor(s) 512 executing the initiator 524 selectively causes the administration of the defibrillation shock based on determining that the individual 508 is exhibiting the shockable rhythm and/or based on an input from a user (received, e.g., by the input device(s) 518. In some cases, the processor(s) 512 causes the defibrillation shock to be output at a particular time, which is determined by the processor(s) 512 based on the ECG signal and/or the impedance signal.

In various cases, the memory 514 further includes a timing coordinator 527 that, when executed by the processor(s) 512, causes the processor(s) 512 to identify a timing relationship of electrical shocks in a sequential-shock therapy, as well as to cause the electrical shocks to be output at the timing relationship. In some examples, the electrical shocks are output by the defibrillator 500. In some cases, the processor(s) 512 coordinate timing of electrical shocks administered by both the defibrillator 500 and an additional defibrillator. For example, the processor(s) 512 generate a shock instruction that causes the additional defibrillator to output a secondary electrical shock at a future time. In some cases, the timing coordinator 527, when executed by the processor(s) 512, causes the processor(s) 512 to identify QRS complexes in the ECG and causes the electrical shocks to be synchronized with the QRS complexes. In some examples, the timing relationship is a predetermined timing relationship.

In some implementations, the processor(s) 512 selectively activate a protection circuit 529 in response to executing at least some instructions in the timing coordinator 527. For example, the processor(s) 512 connect the protection circuit 259 to a path between the ECG port 502 and the detection circuit 510 in response to activating a multi-shock mode. The protection circuit 259, for instance, includes one or more diodes that prevent current from being induced in the detection circuit 510 due to administration of an electrical shock to the individual 507 by another defibrillator coupled with the individual 507. In some cases, the processor(s) 512 deactivates the input device(s) 518 and/or the output device(s) 520 when the defibrillator 500 is in the multi-shock mode.

The processor(s) 512 is operably connected to a charging circuit 523 and a discharge circuit 525. In various implementations, the charging circuit 523 includes a power source 526, one or more charging switches 528, and one or more capacitors 530. The power source 526 includes, for instance, a battery. The processor(s) 512 initiates an electrical shock by causing the power source 526 to charge at least one capacitor among the capacitor(s) 530. For example, the processor(s) 512 activates at least one of the charging switch(es) 528 in the charging circuit 523 to complete a first circuit connecting the power source 526 and the capacitor to be charged. Then, the processor(s) 512 causes the discharge circuit 525 to discharge energy stored in the charged capacitor across a pair of defibrillation electrodes 534, which are in contact with the individual 508. For example, the processor(s) 512 deactivates the charging switch(es) 528 completing the first circuit between the capacitor(s) 530 and the power source 526 and activates one or more discharge switches 532 completing a second circuit connecting the charged capacitor 530 and at least a portion of the individual 508 disposed between defibrillation electrodes 534. In some cases, an electrical shock is administered to the individual 507 by partially discharging the capacitor(s) 530.

The energy is discharged from the defibrillation electrodes 534 in the form of an electrical shock. For example, the defibrillation electrodes 534 are connected to the skin of the individual 508 and located at positions on different sides of the heart of the individual 508, such that the electrical shock is applied across the heart of the individual 508. In some cases, the electrical shock has a multiphasic (e.g., biphasic) waveform. In some examples, a sequence of multiple electrical shocks can prevent the individual 507 from developing an arrhythmia, such as VF. The discharge switch(es) 532 are controlled by the processor(s) 512, for example. In various implementations, the defibrillation electrodes 534 are connected to defibrillation wires 536. The defibrillation wires 536 are connected to a defibrillation port 538, in implementations. According to various examples, the defibrillation wires 536 are removable from the defibrillation port 538. For example, the defibrillation wires 536 are plugged into the defibrillation port 538.

In various implementations, the processor(s) 512 is operably connected to one or more transceivers 540 that transmit and/or receive data over one or more communication networks 542. For example, the transceiver(s) 540 includes a network interface card (NIC), a network adapter, a local area network (LAN) adapter, or a physical, virtual, or logical address to connect to the various external devices and/or systems. In various examples, the transceiver(s) 540 includes any sort of wireless transceivers capable of engaging in wireless communication (e.g., radio frequency (RF) communication). For example, the communication network(s) 542 includes one or more wireless networks that include a 3^{rd} Generation Partnership Project (3GPP) network, such as a Long Term Evolution (LTE) radio access network (RAN) (e.g., over one or more LTE bands), a New Radio (NR) RAN (e.g., over one or more NR bands), or a combination thereof. In some cases, the transceiver(s) 540 includes other wireless modems, such as a modem for engaging in WI-FI®, WIGIG®, WIMAX®, BLUETOOTH®, or infrared communication over the communication network(s) 542.

The defibrillator 500 is configured to transmit and/or receive data (e.g., ECG data, impedance data, data indicative of one or more detected heart rhythms of the individual 508, data indicative of one or more defibrillation shocks administered to the individual 508, etc.) with one or more external devices 544 via the communication network(s) 542. The external devices 544 include, for instance, mobile devices (e.g., mobile phones, smart watches, etc.), Internet of Things (IoT) devices, medical devices (e.g., a primary defibrillator or secondary defibrillator), computers (e.g., laptop devices, servers, etc.), or any other type of computing device configured to communicate over the communication network(s) 542. In some examples, the external device(s) 544 is located remotely from the defibrillator 500, such as at a remote clinical environment (e.g., a hospital). According to various implementations, the processor(s) 512 causes the transceiver(s) 540 to transmit data to the external device(s) 544. In some cases, the transceiver(s) 540 receives data from the external device(s) 544 and the transceiver(s) 540 provide the received data to the processor(s) 512 for further analysis. In some cases, the defibrillator 500 is configure to transmit or receive a shock instruction via the communication network(s) 542.

In various implementations, the external defibrillator 500 also includes a housing 546 that at least partially encloses other elements of the external defibrillator 500. For example, the housing 546 encloses the detection circuit 510, the processor(s) 512, the memory 514, the charging circuit 523, the transceiver(s) 540, or any combination thereof. In some cases, the input device(s) 518 and output device(s) 520 extend from an interior space at least partially surrounded by the housing 546 through a wall of the housing 546. In various examples, the housing 546 acts as a barrier to moisture, electrical interference, and/or dust, thereby protecting various components in the external defibrillator 500 from damage.

In some implementations, the external defibrillator 500 is an automated external defibrillator (AED) operated by an untrained user (e.g., a bystander, layperson, etc.) and can be operated in an automatic mode. In automatic mode, the processor(s) 512 automatically identifies a rhythm in the ECG signal, makes a decision whether to administer a defibrillation shock, charges the capacitor(s) 530, discharges the capacitor(s) 530, or any combination thereof. In some cases, the processor(s) 512 controls the output device(s) 520 to output (e.g., display) a simplified user interface to the untrained user. For example, the processor(s) 512 refrains from causing the output device(s) 520 to display a waveform of the ECG signal and/or the impedance signal to the untrained user, in order to simplify operation of the external defibrillator 500.

In some examples, the external defibrillator 500 is a monitor-defibrillator utilized by a trained user (e.g., a clinician, an emergency responder, etc.) and can be operated in a manual mode or the automatic mode. When the external defibrillator 500 operates in manual mode, the processor(s) 512 cause the output device(s) 520 to display a variety of information that may be relevant to the trained user, such as waveforms indicating the ECG data and/or impedance data, notifications about detected heart rhythms, and the like.

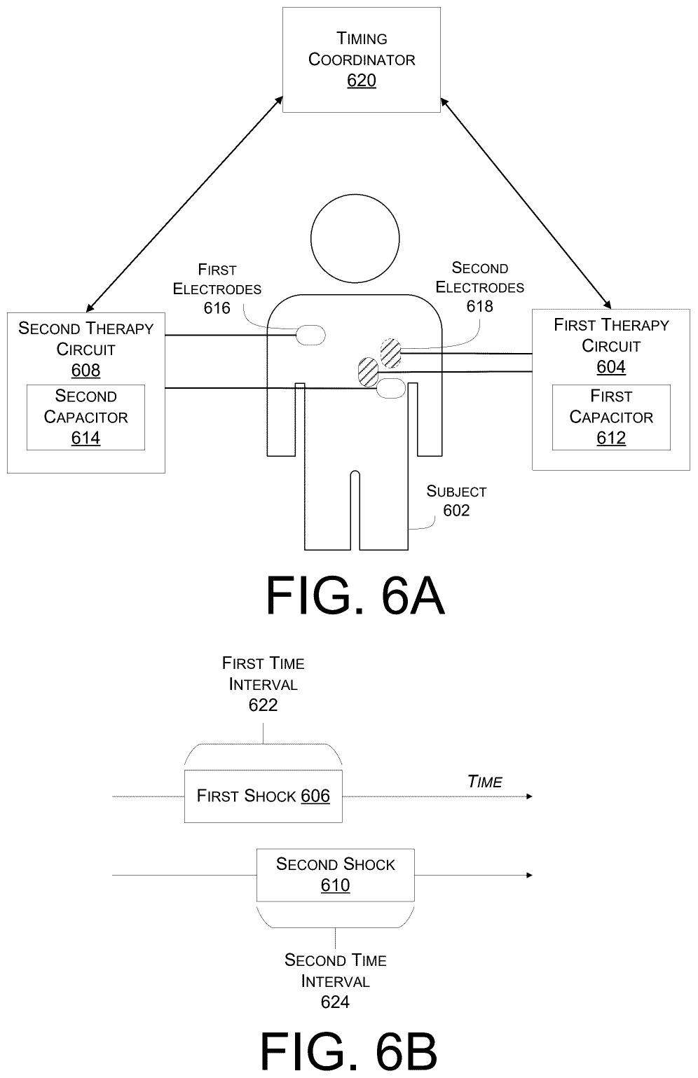

FIGS. 6A and 6B illustrate examples of environments and timing related to administering a sequential-shock therapy. FIG. 6A shows an environment configured to administer the sequential-shock therapy. FIG. 6B shows a timing relationship of multiple shocks administered in the sequential-shock therapy.

In various cases, a subject 602 is predicted to have an arrhythmia that can be treated and/or prevented by the administration of a sequential-shock therapy. Specifically, a first therapy circuit 604 is configured to output a first shock 606 to the subject 602 and a second therapy circuit 608 is configured to output a second shock 610 to the subject 602. In various cases, the first shock 606 and the second shock 610 temporally overlap in time, at least partially. For example, a start time of the second shock 610 occurs after the start time of the first shock 606, but the start time of the second shock 610 occurs before the end time of the first shock 606. In various cases, the first shock 606 is a biphasic shock and/or the second shock 610 is a biphasic shock. In some implementations, the first shock 606 is a monophasic shock and/or the second shock 610 is a biphasic shock. In some cases, the first shock 606 has a shorter duration and/or lower voltage amplitude than the second shock 610.

The first therapy circuit 604 outputs the first shock 606 by discharging a first capacitor 612. Similarly, the second therapy circuit 608 outputs the second shock 610 by discharging a second capacitor 614. In various implementations, one or more power sources are configured to charge the first capacitor 612 and/or the second capacitor 614 prior to discharge. In various cases, the first therapy circuit 604 includes a first H-bridge circuit including the first capacitor 612 and/or the second therapy circuit 608 includes a second H-bridge circuit including the second capacitor 614. The first H-bridge circuit and the second H-bridge circuit are configured to output the first shock 606 and the second shock 610 as biphasic shocks, for instance, via sequential activation of switches in the first H-bridge circuit and the second H-bridge circuit.

The first therapy circuit 604 is configured to output the first shock 606 to first electrodes 616. The second therapy circuit 608 is configured to output the second shock 610 to second electrodes 618. In various cases, the first electrodes 616 and/or the second electrodes 618 are disposed externally on the skin of the subject 602. For example, the first electrodes 616 and/or the second electrodes 618 are adhered to the skin of the subject 602. In various implementations, the first electrodes 616 and the second electrodes 618 are associated with different shock vectors. For instance, a first shock vector extends between the first electrodes 616 and a second shock vector extends between the second electrodes 618, wherein the first shock vector and the second shock vector are different. For example, the first shock vector may be an anterior-lateral position and the second shock vector may be an anterior-posterior position. In various implementations, the first shock vector and the second shock vector both extend through the heart of the subject 602. Although FIG. 6A illustrates the first electrodes 616 as being separate from the second electrodes 618, implementations are not so limited. For example, one electrode may be shared among the first electrodes 616 and the second electrodes 618.

The first therapy circuit 604 and the second therapy circuit 608 are distributed among one or more devices. In some cases, the first therapy circuit 604 is part of a first external defibrillator and the second therapy circuit 608 is part of a second external defibrillator. For example, the first external defibrillator and the second external defibrillator are both monitor-defibrillators, both AEDs, or a monitor-defibrillator and an AED. In some cases, the first therapy circuit 604 or the second therapy circuit 608 is integrated into an accessory device without monitoring capabilities, and which is solely designed to output electrical shocks upon receiving an input signal from a separate device. For example, the accessory device may lack, or be disconnected from, one or more sensors configured to identify one or more physiological parameters of the subject 602. In some cases, the accessory device lacks a display, speaker, or other user interface device. In some implementations, the first therapy circuit 604 and the second therapy circuit 608 are integrated into the same device, such as the same monitor-defibrillator.

Optionally, a timing coordinator 620 is configured to cause the first therapy circuit 604 to output the first shock 606 during a first time interval 622 and/or to cause the second therapy circuit 608 to output the second shock 610 during the second time interval 624. For example, the timing coordinator 620 outputs one or more signals (e.g., electrical signals, communication signals, etc.) to the first therapy circuit 604 and/or the second therapy circuit 608. Upon receiving the signal(s) from the timing coordinator 620, the first therapy circuit 604 may discharge the first capacitor 612 during the first time interval 622 and/or the second therapy circuit 608 may discharge the second capacitor 614 during the second time interval 624. The timing coordinator 620 can be implemented in hardware (e.g., a circuit), software (e.g., instructions executed by at least one processor), or a combination thereof. In some cases, the timing coordinator 620 is a standalone device. Examples of standalone timing devices that can serve as the timing coordinator 620 are described in U.S. Pat. No. 10,981,014, which is incorporated by reference herein in its entirety. In some examples, the timing coordinator 620 is integrated into the same device as the first therapy circuit 604 and/or the second therapy circuit 608.

Various timing relationships between the first time interval 622 and the second time interval 624 can be implemented according to various implementations of the present disclosure. In some cases, a delay between the start times (i.e., the leading edges) of the first time interval 622 and the second time interval 624 is in a range of 0 and 250 milliseconds (ms). In some cases, the delay between the start times of the first time interval 622 and the second time interval 624 is in a range of −250 and 0 ms. Although FIG. 6B illustrates the first time interval 622 and the second time interval 624 as having equivalent durations, implementations are not so limited. For example, the first time interval 622 may be longer or shorter than the second time interval 624. Various timing relationships are described in U.S. Pat. No. 10,702,701, which is incorporated by reference herein in its entirety.

In various cases, the timing coordinator 620 is configured to detect the first shock 606 and may cause the second therapy circuit 608 to output the second shock 610 in response. For example, the timing coordinator 620 may detect a signal indicative of the discharge of the first shock 606, and may output a signal that causes the second therapy circuit 608 to discharge the second shock 610. In some examples, the timing coordinator 620 is inductively coupled with the first therapy circuit 604 and/or the first electrodes 616, which enables the timing coordinator 620 to detect the discharge of the first shock 606. Various techniques for detecting the discharge of a first shock in order to cause the application of a second shock in sequential-shock therapy are described in U.S. Pat. Nos. 10,625,088 and 10,632,320, which are incorporated by reference herein in their entirety.

### Example Clauses

The following example clauses provide various implementations of the present disclosure. However, the scope of the present disclosure is not limited to any of the example clauses presented herein.
1. A defibrillator, including: a measurement circuit configured to detect an electrocardiogram (ECG) of a subject by detecting a voltage across electrodes configured to be disposed on skin of the subject; a treatment circuit including a capacitor; and a processor configured to: determine, by analyzing a segment of the ECG that is not indicative of ventricular fibrillation (VF), that the subject is predicted to develop VF; and in response to determining that the subject is predicted to develop VF, prevent the subject from developing VF by causing the treatment circuit to output a sequence of external shocks to a heart of the subject by: outputting a first external shock by partially discharging the capacitor; and outputting a second external shock by partially discharging the capacitor.
2. The defibrillator of clause 1, further including: a transceiver configured to output, to another defibrillator, an instruction to output a third external shock that temporally overlaps with the first external shock or the second external shock.
3. The defibrillator of clause 1 or 2, further including: a display configured to output a recommendation to administer the sequence of external shocks; and an input device configured to receive an input signal from a user, wherein the processor is further configured to: in response to determining that the subject is predicted to develop VF, cause the display to output the recommendation to administer the sequence of external shocks, and in response to the input device receiving the input signal, cause the treatment circuit to output the sequence of external shocks to the heart of the subject.
4. A method, including: determining, before a subject has developed an arrhythmia or recurrence of the arrhythmia, that the subject is predicted to develop the arrhythmia; and in response to determining that the subject is predicted to develop the arrhythmia, administering a sequence of external shocks to a heart of the subject, thereby preventing the subject from developing the arrhythmia or the recurrence of the arrhythmia.
5. The method of clause 4, wherein the arrhythmia includes ventricular fibrillation (VF) or ventricular tachycardia (VT).
6. The method of clause 4 or 5, wherein the arrhythmia includes at least one of a premature ventricular contraction (PVC), atrial fibrillation, or supraventricular tachycardia.
7. The method of any of clauses 4 to 6, wherein determining, before the subject has developed the arrhythmia or recurrence of the arrhythmia, that the subject is predicted to develop the arrhythmia includes: determining that an ECG of the subject is indicative of coarse VF, STEMI, OMI, atrial fibrillation, supraventricular tachycardia, excessive PVCs, insufficient PVCs, or an AV block.
8. The method of any of clauses 4 to 7, wherein determining, before the subject has developed the arrhythmia or recurrence of the arrhythmia, that the subject is predicted to develop the arrhythmia includes: determining that the subject has a condition associated with a susceptibility to the arrhythmia.
9. The method of clause 8, wherein determining that the subject has the condition includes identifying the condition based on a patient history of the subject or based on a physiological parameter of the subject.
10. The method of clause 8 or 9, wherein the condition includes Wolf-Parkinson-White syndrome.
11. The method of clause any of clauses 4 to 10, wherein a number of external shocks in the sequence of external shocks is in a range of 2 to 10.
12. The method of any of clauses 4 to 11, wherein the external shocks are administered at multiple energy levels.
13. The method of any of clauses 4 to 12, wherein the external shocks are administered at multiple vectors.
14. The method of any of clauses 4 to 13, wherein an external shock among the sequence of external shocks has an energy level in a range of about 50 to about 360 J.
15. The method of any of clauses 4 to 14, wherein a delay between a leading edge of a first external shock among the sequence of external shocks and a leading edge of a second external shock among the sequence of external shocks is in a range of about 10 ms to about 10,000 ms.
16. The method of any of clauses 4 to 15, wherein: the sequence of external shocks are administered by a first defibrillator and a second defibrillator, and administering the sequence of external shocks to the heart of the subject includes: outputting, by the first defibrillator, a first external shock among the sequence of external shocks; outputting, by the second defibrillator, a second external shock among the sequence of external shocks; and outputting, by the first defibrillator, a third external shock among the sequence of external shocks.
17. The method of clause 16, further including: in response to outputting, by the first defibrillator, the first external shock among the sequence of external shocks, recharging a capacitor of the first defibrillator, wherein outputting, by the first defibrillator, the third external shock is in response to recharging the capacitor of the first defibrillator.
18. The method of clause 16 or 17, wherein the first defibrillator or the second defibrillator includes a wearable defibrillator.
19. The method of any of clauses 4 to 18, wherein the sequence of external shocks are administered by a single defibrillator.
20. The method of clause 19, wherein administering the sequence of external shocks to the heart of the subject includes: outputting, by the single defibrillator, a first external shock by partially discharging a capacitor of the single defibrillator; and in response to outputting the first external shock, outputting, by the single defibrillator, a second external shock by fully discharging the capacitor.
21. The method of clause 19 or 20, wherein administering the sequence of external shocks to the heart of the subject includes: outputting, by the single defibrillator, a first external shock by fully discharging a capacitor of the single defibrillator; in response to outputting the first external shock, charging the capacitor; and in response to charging the capacitor, outputting, by the single defibrillator, a second external shock by discharging the capacitor.
22. The method of any of clauses 4 to 21, further including: determining that an ECG of the subject includes QRS complexes, wherein administering the sequence of external shocks to the heart of the subject includes administering the sequence of external shocks synchronized with QRS complexes of the subject.
23. The method of any of clauses 4 to 22, wherein administering the sequence external shocks to the heart of the subject includes administering one of the external shocks during a release phase of chest compressions administered to the subject.
24. The method of any of clauses 4 to 23, wherein administering the sequence of external shocks to the heart of the subject includes administering one of the external shocks during a compression phase of chest compressions administered to the subject.
25. The method of any of clauses 4 to 24, further including: determining that an ECG of the subject lacks QRS complexes, wherein the sequence of external shocks have a predetermined timing relationship.
26. The method of any of clauses 4 to 25, further including: determining that the subject is unconscious, wherein administering the sequence of external shocks to the heart of the subject is further in response to determining that the subject is unconscious.
27. The method of any of clauses 4 to 26, further including: outputting a recommendation to administer the sequence of external shocks to the heart of the subject; and receiving, from a user, an input signal, wherein administering the sequence of external shocks to the heart of the subject is further in response to receiving the input signal.
28. A medical device, including: a measurement circuit configured to detect a physiological parameter of a subject; and a processor configured to: determine, by analyzing the physiological parameter before the subject has developed an arrhythmia or recurrence of the arrhythmia, that the subject is predicted to develop the arrhythmia; and in response to determining that the subject is predicted to develop the arrhythmia, outputting a recommendation to administer a sequence of external shocks to a heart of the subject.
29. The medical device of clause 28, wherein the processor is configured to determine that the subject is predicted to develop the arrhythmia further based on a demographic or patient history of the subject.
30. The medical device of clause 28 or 29, wherein the physiological parameter includes an ECG, a blood flow, or a transthoracic impedance.
31. The medical device of any of clauses 28 to 30, wherein the arrhythmia includes VF or VT.
32. The medical device of clause 31, wherein the arrhythmia includes at least one of a premature ventricular contraction (PVC), atrial fibrillation, or supraventricular tachycardia.
33. The medical device of any of clauses 28 to 32, wherein the processor is configured to determine, by analyzing the physiological parameter before the subject has developed the arrhythmia or recurrence of the arrhythmia, that the subject is predicted to develop the arrhythmia by: determining that the physiological parameter of the subject is indicative of coarse VF, STEMI, OMI, atrial fibrillation, supraventricular tachycardia, excessive PVCs, insufficient PVCs, or an AV block.
34. The medical device of any of clauses 28 to 33, wherein the processor is configured to determine, by analyzing the physiological parameter before the subject has developed the arrhythmia or recurrence of the arrhythmia, that the subject is predicted to develop the arrhythmia by: determining that the subject has a condition associated with a susceptibility to the arrhythmia.
35. The medical device of any of clauses 28 to 34, wherein a number of external shocks in the sequence of external shocks is in a range of 2 to 10.
36. The medical device of any of clauses 28 to 35, wherein the external shocks are administered at multiple energy levels.
37. The medical device of any of clauses 28 to 36, wherein the external shocks are administered at multiple vectors.
38. The medical device of any of clauses 28 to 37, wherein an external shock among the sequence of external shocks has an energy level in a range of about 50 to about 360 J.
39. The medical device of any of clauses 28 to 38, wherein a delay between a leading edge of a first external shock among the sequence of external shocks and a leading edge of a second external shock among the sequence of external shocks is in a range of about 10 to about 10,000 ms.
40. The medical device of any of clauses 28 to 39, further including: a treatment circuit configured to output, to the subject, a first external shock among the sequence of external shocks.
41. The medical device of clause 40, wherein the sequence of external shocks further includes a second external shock among the sequence of external shocks, the second external shock being output by a defibrillator that is separate from the medical device.
42. The medical device of clause 40 or 41, wherein: the treatment circuit includes a capacitor and a power source, and the treatment circuit is configured to: output the first external shock by discharging the capacitor; in response to outputting the first external shock, recharging the capacitor using the power source; and in response to recharging the capacitor, outputting a second external shock by discharging the capacitor.
43. The medical device of any of clauses 40 to 42, wherein: the treatment circuit includes a capacitor, and the treatment circuit is configured to: output the first external shock by partially discharging the capacitor; and in response to outputting the first external shock, output a second external shock by partially discharging the capacitor.
44. The medical device of any of clauses 28 to 43, wherein the medical device is a wearable defibrillator.
45. The medical device of any of clauses 28 to 44, wherein the processor is further configured to: determine that an ECG of the subject includes QRS complexes, wherein the recommendation to administer the sequence of external shocks to the heart of the subject includes an instruction to administer the sequence of external shocks synchronized with QRS complexes of the subject.
46. The medical device of any of clauses 28 to 45, wherein the recommendation to administer the sequence of external shocks to the heart of the subject includes an instruction to administer one of external shocks during a release phase of chest compressions administered to the subject.
47. The medical device of any of clauses 28 to 46, wherein the recommendation to administer the sequence of external shocks to the heart of the subject includes an instruction to administer one of the external shocks during a compression phase of chest compressions administered to the subject.
48. The medical device of any of clauses 28 to 47, wherein the processor is further configured to determine that an ECG of the subject lacks QRS complexes, wherein the recommendation to administer the sequence of external shocks indicates a predetermined timing relationship of the sequence of external shocks.
49. The medical device of any of clauses 28 to 48, wherein the processor is further configured to: determine that the subject is unconscious, wherein outputting the recommendation to administer the sequence of external shocks to the heart of the subject is further in response to determining that the subject is unconscious.
50. The medical device of clause 49, wherein the processor is configured to determine that the subject is unconscious by detecting an absence of an irregular motion artifact in the physiological parameter.
51. The medical device of clause 49 or 50, further including: a movement sensor configured to detect a movement of the subject, wherein the processor is configured to determine that the subject is unconscious by analyzing the movement of the subject.
52. The medical device of any of clauses 28 to 51, further including: an output device configured to output the recommendation.

### Conclusion

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about. ” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain implementations are described herein, including the best mode known to the inventors for carrying out implementations of the disclosure. Of course, variations on these described implementations will become apparent to those of ordinary skill in the art upon reading the foregoing description. The inventors expect skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

What is claimed is:

1. A defibrillator, comprising:
a measurement circuit configured to detect an electrocardiogram (ECG) of a subject by detecting a voltage across electrodes configured to be disposed on skin of the subject;
a treatment circuit comprising a capacitor; and
a processor configured to:
determine, by analyzing a segment of the ECG that is not indicative of ventricular fibrillation (VF), that the subject is predicted to develop VF; and
in response to determining that the subject is predicted to develop VF, prevent the subject from developing VF by causing the treatment circuit to output a sequence of external shocks to a heart of the subject by:
outputting a first external shock by partially discharging the capacitor; and
outputting a second external shock by partially discharging the capacitor.

2. The defibrillator of claim 1, further comprising:
a transceiver configured to output, to another defibrillator, an instruction to output a third external shock that temporally overlaps with the first external shock or the second external shock.

3. The defibrillator of claim 1, further comprising:
a display configured to output a recommendation to administer the sequence of external shocks; and
an input device configured to receive an input signal from a user,
wherein the processor is further configured to:
in response to determining that the subject is predicted to develop VF, cause the display to output the recommendation to administer the sequence of external shocks, and
in response to the input device receiving the input signal, cause the treatment circuit to output the sequence of external shocks to the heart of the subject.

4. A method, comprising:
determining, before a subject has developed an arrhythmia or recurrence of the arrhythmia, that the subject is predicted to develop the arrhythmia; and
in response to determining that the subject is predicted to develop the arrhythmia, administering a sequence of external shocks to a heart of the subject, thereby preventing the subject from developing the arrhythmia or the recurrence of the arrhythmia.

5. The method of claim 4, wherein the arrhythmia comprises at least one of VF, VT, a premature ventricular contraction (PVC), atrial fibrillation, or supraventricular tachycardia.

6. The method of claim 4, wherein determining, before the subject has developed the arrhythmia or recurrence of the arrhythmia, that the subject is predicted to develop the arrhythmia comprises:
determining that an ECG of the subject is indicative of coarse VF, STEMI, OMI, atrial fibrillation, supraventricular tachycardia, excessive PVCs, insufficient PVCs, or an AV block.

7. The method of claim 4, wherein a number of external shocks in the sequence of external shocks is in a range of 2 to 10.

8. The method of claim 4, wherein the external shocks are administered at multiple energy levels or at multiple vectors.

9. The method of claim 4, wherein:
the sequence of external shocks are administered by a first defibrillator and a second defibrillator, and
administering the sequence of external shocks to the heart of the subject comprises:
outputting, by the first defibrillator, a first external shock among the sequence of external shocks;
in response to outputting, by the first defibrillator, the first external shock among the sequence of external shocks, recharging a capacitor of the first defibrillator;
outputting, by the second defibrillator, a second external shock among the sequence of external shocks; and
in response to recharging the capacitor of the first defibrillator, outputting, by the first defibrillator, a third external shock among the sequence of external shocks.

10. The method of claim 9, wherein the first defibrillator or the second defibrillator comprises a wearable defibrillator.

11. The method of claim 4, wherein the sequence of external shocks are administered by a single defibrillator, and
wherein administering the sequence of external shocks to the heart of the subject comprises:
outputting, by the single defibrillator, a first external shock by partially discharging a capacitor of the single defibrillator; and
in response to outputting the first external shock, outputting, by the single defibrillator, a second external shock by fully discharging the capacitor.

12. The method of claim 4, further comprising:
determining that an ECG of the subject comprises QRS complexes,
wherein administering the sequence of external shocks to the heart of the subject comprises administering the sequence of external shocks synchronized with QRS complexes of the subject.

13. The method of claim 4, wherein administering the sequence external shocks to the heart of the subject comprises administering one of the external shocks during a release phase of chest compressions administered to the subject.

14. The method of claim 4, wherein administering the sequence of external shocks to the heart of the subject comprises administering one of the external shocks during a compression phase of chest compressions administered to the subject.

15. The method of claim 4, further comprising:
determining that the subject is unconscious,
wherein administering the sequence of external shocks to the heart of the subject is further in response to determining that the subject is unconscious.

16. A medical device, comprising:
a measurement circuit configured to detect a physiological parameter of a subject; and
a processor configured to:
determine, by analyzing the physiological parameter before the subject has developed an arrhythmia or recurrence of the arrhythmia, that the subject is predicted to develop the arrhythmia; and
in response to determining that the subject is predicted to develop the arrhythmia, outputting a recommendation to administer a sequence of external shocks to a heart of the subject.

17. The medical device of claim 16, wherein the physiological parameter comprises an ECG, a blood flow, or a transthoracic impedance.

18. The medical device of claim 16, further comprising:
a treatment circuit configured to output, to the subject, a first external shock among the sequence of external shocks.

19. The medical device of claim 18, wherein:
the treatment circuit comprises a capacitor, and
the treatment circuit is configured to:
output the first external shock by partially discharging the capacitor; and
in response to outputting the first external shock, output a second external shock by partially discharging the capacitor.

20. The medical device of claim 16, wherein the processor is further configured to:
determine that the subject is unconscious by detecting an absence of an irregular motion artifact in the physiological parameter,
wherein outputting the recommendation to administer the sequence of external shocks to the heart of the subject is further in response to determining that the subject is unconscious.
